(19) United States
(12) Patent Application Publication
(10) Pub. No.: US 2026/0229310 A1
(43) Pub. Date: Aug. 6, 2026

(54) System and Method for Multiomics Assessment of Capillary Blood over Space and Time

(71) Applicant: Mohammed Harris, Basking Ridge, NJ (US)

(72) Inventor: Mohammed Harris, Basking Ridge, NJ (US)

(73) Assignee: TimepointDx, Basking Ridge, NJ (US)

(21) Appl. No.: 19/046,062

(22) Filed: Feb. 5, 2025

(51) Int. Cl. G16B 25/10; C12Q 1/6869; G16B 20/20; G16H 15/00; G16H 20/00; G16H 50/20; G16H 50/30; G16H 50/70

(52) U.S. Cl. CPC G16B 25/10; C12Q 1/6869; G16B 20/20; G16H 15/00; G16H 20/00; G16H 50/20; G16H 50/30; G16H 50/70

(57) ABSTRACT

The disclosure provides a system and method for longitudinal multiomics assessments of capillary blood microsamples collected from the upper-arm using a push-button blood collection device, whereby collected samples are subjected to simultaneous, parallel, sequential and longitudinal analysis of genomics (via whole genome sequencing, whole exome sequencing, and aggregated longitudinal sequencing), transcriptomics (via bulk RNA sequencing), and proteomics (via oligonucleotide-incorporated or oligonucleotide-coupled labeling and detection proteomics platforms) to provide a holistic view of biological systems and biomarkers and their interactions over space (spatial) and time (temporal). The disclosure also provides a practical workflow and computational framework for integration and processing of longitudinal capillary multiomics (LCM) data in the contexts of clinical trials, primary healthcare, and population-level studies.

### FIELD OF THE INVENTION

The present invention relates to a system and method for multiomics assessment of capillary blood samples. The invention enables Longitudinal Capillary Multiomics (LCM) analysis by integrating genomics, transcriptomics, and proteomics across spatial and temporal domains, offering real-time insights into biological changes over time.

### BACKGROUND OF THE INVENTION

Longitudinal multiomics is a cutting-edge approach observing different layers of biological data including genomics, transcriptomics, proteomics, and metabolomics change over time. By tracking these changes at multiple timepoints, researchers can uncover greater understanding of the dynamic nature of biological systems. This approach is particularly valuable in studying complex processes like disease development, aging and how the environment influences our biology. Unlike traditional cross-sectional studies offering only a glimpse of data at a single moment, longitudinal studies via analyzing time-series data for a plurality of variables reveal molecular signatures and how they evolve, leading to a better understanding of disease progression and treatment responses.

LCM captures temporal changes in genetic mutations, transcriptional activity and protein expression, offering unique insights into disease progression, treatment response, biomarker discovery, and therapeutic outcomes. By linking molecular profiles to clinical outcomes, multiomics approaches have demonstrated their potential to uncover disease mechanisms and advance precision health.

### PRIOR ART

Several published prior arts dealing with capillary blood sample collection along with diagnostic applications in several domains are illustrated hereinafter.

U.S. Pat. No. 8,808,202B2 titled, “Systems and interfaces for blood sampling” generally relates to systems and methods for delivering and/or receiving a substance or substances such as blood from subjects. In one aspect, the invention is directed to devices and methods for receiving or extracting blood from a subject, e.g., from the skin and/or from beneath the skin, using devices containing a substance transfer component (for example, one or more needles or microneedles) and a reduced pressure or vacuum chamber having an internal pressure less than atmospheric pressure prior to receiving blood. In some embodiments, the device may contain a “snap dome” or other deformable structure, which may be used, at least in part, to urge or move needles or other suitable substance transfer components into the skin of a subject. In some cases, for example, the device may contain a flexible concave member and a needle mechanically coupled to the flexible concave member.

U.S. Pat. No. 11,932,907B2 titled, “Fetal sex determination using capillary blood from upper arm” mentions of a disclosure that relate to methods, compositions, and kits for the early determination of the sex of a fetus. The disclosure also provides methods, compositions, and kits for detecting fetal nucleic acids in biological samples (e.g., cell-free fetal DNA). An embodiment includes a method of improving the accuracy of fetal sex determination by reducing a level of contaminating DNA in a blood sample from a pregnant human subject, comprising obtaining a capillary blood sample collected from the upper arm using a push-button blood collection device, thereby reducing a level of contaminating DNA from a non-maternal and non-fetal source in the capillary blood sample as compared to a blood sample collected from a site on the finger or hand of the subject, and detecting the presence or absence of fetal Y-chromosome to determine the sex of the fetus.

US20240011075A1 titled, “Immune profiling using small volume blood samples” discloses embodiments of a method for single cell ribonucleic acid sequencing. In some embodiments, the method comprises providing a first low volume, capillary blood sample (or any low volume blood sample and/or any blood sample not obtained from a vein or by venipuncture) obtained from a subject at a first time point. The method can comprise diluting the first sample to obtain a first diluted sample. The method can comprise isolating first capillary peripheral blood mononuclear cells (cPBMCs) from the first diluted sample with gradient centrifugation. The method can comprise performing single cell ribonucleic acid sequencing (scRNA-seq) on the first cPBMCs isolated to generate first scRNA-seq data. The method can comprise determining a first scRNA profile of the subject at the first time point using the first scRNA-seq data and single-nucleotide polymorphisms (SNPs) of the subject.

However, in none of these inventions, the collection of such blood sample from the upper arm using specialized devices along with LCM assessments of the samples for longitudinal and sequential time periods for the creation of temporal and spatial data has been incorporated.

The present invention embodies both the stated aspects in the analyses of capillary blood samples towards precise evaluation of periodic samples in a longitudinal manner, whereby data can be aggregated and cumulative over time.

### SUMMARY OF THE INVENTION

An aspect of the invention is a system and method for longitudinal multiomics assessments of capillary blood samples.

Another aspect of the invention allows all samples being collected in microvials (e.g. BD Microtainer) on account of the sample collection being in micro volume.

Further aspect of the invention allows the samples to be collected anywhere (remote, at-home, point of care), i.e., spatial as well as over time, i.e., temporal.

In another aspect of the invention, capillary blood samples are specifically collected as liquid capillary whole blood collections through upper arm blood collection devices.

In an aspect of the invention, such assessments of LCM are undertaken through genomics, transcriptomics and proteomics analyses towards biomarker evaluation of blood samples.

Another aspect of the invention involves the LCM technology to have other multiomics assay, e.g., metabolomics, epigenomics, exposomics, lipidomics and other omics of the blood samples.

In further aspect of the invention, LCM is a time-based assay, ensuring a longitudinal aspect in utility and enabling longitudinal “timepoints” to create a time-series of sample collections that are assessed by the LCM technology so as to create a biomarker picture of a typical person from whom samples are collected at various timepoints.

Another aspect of the invention is to undergo LCM analysis through Whole Genome Sequencing (WGS) and Whole Exome Sequencing (WES) for genomics, bulk RNA sequencing (bRNAseq) for transcriptomics and oligonucleotide-incorporated and oligonucleotide-coupled platforms for proteomics.

In an aspect of the invention, Whole Genome Sequencing (WGS) provides a snapshot of the entire genome for germline aberrations, leading to greater understanding of the whole genetic architecture, whereas Whole Exome Sequencing (WES) provides a more detailed view of exome coding regions over time for possible somatic genetic aberrations.

Another aspect of the invention is the “accumulative” or “combinatorial” or “additive” or “aggregative” sequencing feature of the genomic component of the LCM technique deployed, whereby low-pass sequencing (e.g., low-pass WGS) and high-pass sequencing (e.g., high-pass WES) coverages can be added together over time to create an additive and aggregated effect of sequencing coverage called “Aggregated Longitudinal Sequencing” (ALseq).

In another aspect of the invention, in the conducting of Whole Genome Sequencing (WGS) and Whole Exome Sequencing (WES), a coverage range generally from 0.1× to 100× is maintained, whereby low-pass sequencing is defined as coverages between 0.1× to 10× and high-pass sequencing is defined as coverages greater than 10× (e.g. 30×).

Another aspect of the invention allows bulk mRNA sequencing (bRNAseq) or single-cell RNA sequencing (scRNAseq) being conducted for the transcriptomics assay of the LCM analysis.

In an aspect of the invention, the coupling of oligonucleotides is bound to antibodies or aptamers in the proteomics platforms leading to two leading platforms such as Olink (e.g. Exlore, HT, and Reveal) and SomaLogic (e.g. 7k, 9k, and 11K) proteomics.

Still in another aspect of the invention, apart from Olink and SomaLogic platforms there can be two other similar proteomics platforms, Nomic Bio and Alamar Biosciences platforms, so as not to be limited in context of proteomics, while still incorporating proteomics platforms that have an oligonucleotide component integrated into the detection and coupling chemistries.

In another aspect of the invention, coupling/introduction of oligonucleotides in the proteomics assays is achieved through proximity-based extension (PEA) technology for the Olink platform, aptamer-based technology for the SomaLogic platform, nELISA for the Nomic Bio platform, and NULISA for the Alamar Biosciences platform.

Still further aspect of the invention allows all components of the LCM (i.e. genomics, transcriptomics, and proteomics) to be conducted on the same foundational platform (e.g. Illumina sequencing instruments, Element Biosciences sequencing instruments, Ultima Genomics sequencing instruments, Oxford Nanopore Technologies sequencing instruments) and in some instances may be conducted on the same foundation sequencing flow cell of such foundational platform instruments.

In another aspect of the invention, the ability to conduct genomics, transcriptomics, and proteomics within the same next-generation sequencing platform and within the same sequencing flow cell on the same sequencing run, as a result of combining sequencing libraries across all of the omics is described as combinatorial multiomics libraries, enabling efficiencies for LCM.

Still further aspect of the invention is the accrual of economic benefits in LCM analysis on account of cost minimization besides the ability to assimilate greater statistical power and reproducibility of the collected samples through the use of capillary blood collections and decentralized workflows.

In another aspect of the invention, LCM has primary benefit to be used as an “exploratory biomarker assessments” or “exploratory biomarker endpoints” as formally designated within lab manuals and protocols of clinical trials, whereby incorporation can be in clinical trials stages of preclinical stage, discovery stage, Phase 1, Phase 2, Phase 3, and Phase 4 (post-marketing surveillance).

Further aspect of the invention allows integration of omics data using sophisticated computational tools (advanced statistical tools, e.g., R-Programming ecosystem) for analyzing the vast datasets generated by LCM, unifying complex multiomics data to reveal molecular connections that might be overlooked in single-omics analyses.

In another aspect of the invention, the LCM analysis process is standardized and automated using artificial intelligence (AI) and machine learning (ML) algorithms that converts and weaves raw data from LCM into insightful reports.

Final aspect of the invention allows the LCM technique to be applied in a plurality of areas in the prediction of disease propensity for purposes of preventive health, early detection, active surveillance, and real-time monitoring of biomarker status and health

### BRIEF DESCRIPTION OF THE DRAWINGS

FIG. 1 is a schematic workflow of Longitudinal Capillary Multiomics (LCM) process, whereby capillary whole blood is collected from an upper-arm blood collection device and captured in a microvial for subsequent LCM assay and analysis.

FIG. 2 is a schematic diagram showing the overall general LCM workflow in a simplified box schematic.

FIG. 3 is a schematic diagram showing the longitudinal nature of capillary blood collections over time and space for the LCM workflow, whereby each timepoint undergoes a LCM analysis.

FIG. 4 is a schematic diagram of the various components of the LCM technique, whereby genomics (WGS, WES, and ALseq), transcriptomics (bRNAseq or scRNAseq), and proteomics (Olink, SomaLogic, Nomic Bio, or Alamar Biosciences) are conducted within LCM for every timepoint.

FIG. 5 is a schematic comparison of Aggregated Longitudinal Sequencing (ALseq) for the genomics component of LCM vs. traditional sequencing.

FIG. 6 is a schematic comparison demonstrating the cumulative and additive effects of Aggregated Longitudinal Sequencing (ALseq) in LCM.

FIG. 7 is a schematic diagram of an example of a bioinformatics pipeline for data analysis of Aggregated Longitudinal Sequencing (ALseq) in LCM, whereby the example pipeline shows temporal and spatial aggregation of genomics data within LCM.

FIGS. 8A, 8B, and 8C are schematic diagrams showing examples of primary file formats used in LCM data analysis and example bioinformatics schemas for LCM data integration in parallel, sequential, spatial, and temporal analysis.

FIG. 9 is a schematic diagram that demonstrates the parallel (genomics+transcriptomics+proteomics) and sequential (longitudinal time-series) of the LCM assay and workflow.

FIG. 10 is a schematic depiction of example LCM applications, including but not limited to, exploratory biomarkers analysis in clinical trials, translational research and discovery, primary healthcare diagnostics, public health, population health, community health, cell-free DNA (cfDNA) and circulating tumor DNA (ctDNA) assessments, spatial hematopathology, characterization of in vitro cell cultures, germline mutational rate and somatic mutational rate assessments, morphometric analysis and assessments (e.g. cytogenetics, H&E, AI/ML digital pathology of microscope blood slides and smears), minimal residual disease (MRD) assessments, air quality monitoring with exposomics analysis, gene therapy monitoring assessments, quantitative systems pharmacology (QSP) and modeling and simulation (M&S) assessments, and pharmacokinetics (PK) and pharmacodynamics (PD) assessments.

### DETAILED DESCRIPTION OF THE INVENTION

Various examples of the invention are now described. The following description provides specific details for a thorough understanding of the invention with enabling descriptions. One skilled in the relevant art will understand that the invention may be practiced without many of these details. Likewise, one skilled in the relevant art will also understand that the invention can include many other obvious features not described in detail herein.

Before explaining at least one embodiment of the inventive concepts disclosed herein in detail, it is to be understood that the inventive concepts are not limited in their application to the details of the steps or methodologies set forth in the following description or illustrated in the drawings. The inventive concepts disclosed herein are capable of other embodiments, or of being practiced or carried out in various ways. Also, it is to be understood that the phraseology and terminology employed herein is for the purpose of description and should not be regarded as limiting the inventive concepts disclosed and claimed herein in any way.

In the following detailed description of embodiments of the inventive concepts, numerous specific details are set forth to provide a more thorough understanding of the inventive concepts. However, it will be apparent to one of ordinary skill in the art that the inventive concepts within the disclosure may be practiced without these specific details. In other instances, well-known features have not been described in detail to avoid unnecessarily complicating the instant disclosure.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

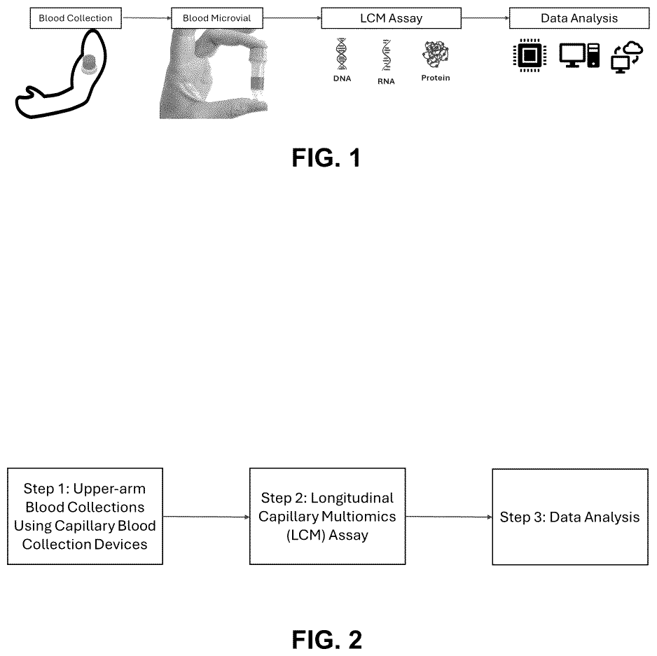

Therefore, according to the present invention, there is provided a system and method for the LCM workflow and process for patient-centric exploratory biomarkers analysis over space and time. With reference to FIGS. 1 and 2, the workflow comprises of three broad steps:
a. Capillary blood sampling method;
b. LCM assessment; and
c. Integration and analyses of LCM data.

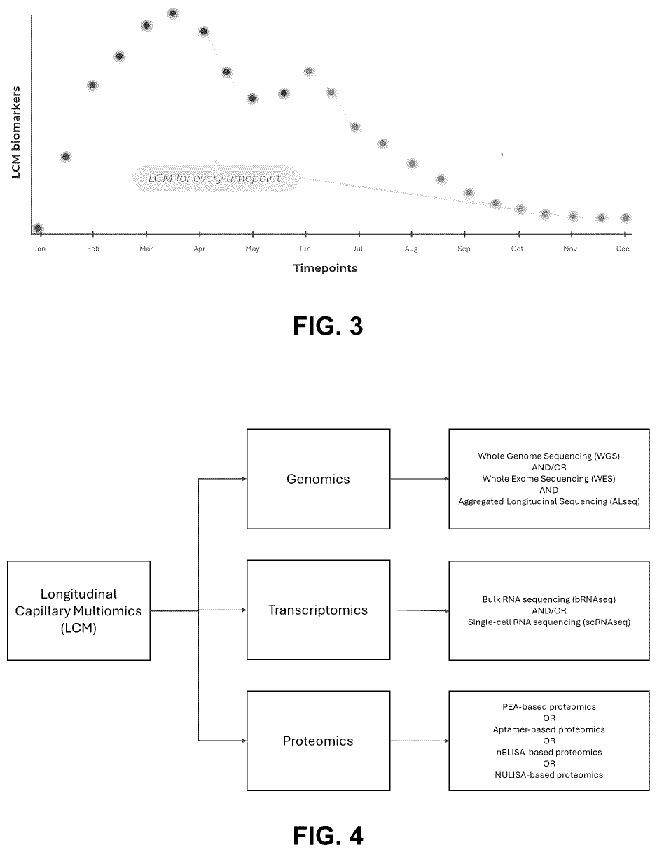

The said workflow at the first instance allows capillary blood samples to be collected from patients in the upper-arm through specialized blood collection devices for the upper arm (e.g. Tasso+, Yourbio Health TAP Micro Select, Reddrop Dx One, and similar devices) (FIGS. 1 AND 2) over periods (t₁, t₂, t₃ . . . ) of time, thereby embodying a longitudinal perspective of such collection procedure. Each of these capillary blood samples are thereupon subjected to LCM assessments, wherein each of these samples are analyzed for Genomics (WGS/WES), transcriptomics (bRNAseq or scRNAseq) and proteomics (e.g. Olink, Somalogic, Nomic Bio, Alamar Biosciences) over time to create a longitudinal multiomics biomarker profile of a person (FIG. 3).

In the following sections, the said LCM process shall be described along with associated implications in detail. Starting with the capillary blood sampling method thereof, a person is subjected to a less invasive and more patient-centric alternative to traditional venous blood drawing wherein, through upper-arm blood collection devices, like Tasso+, Yourbio Health TAP Micro Select, Reddrop Dx One, and similar such devices, blood samples from capillaries in the fingertips or upper arms are collected over various points of time and space. Such a less-invasive approach finds favor with infants, children and elderly, thereby increasing its industrial and commercial applicability and utility.

Further, the said system upon drawing of capillary blood from the subject for multiple times over a period of time entails processing of the samples through LCM analyses (FIG. 4) to extract genomic, transcriptomic and proteomic data and analyzing these towards understanding of their changes over time for the purposes of biomarker health assessments. The genomics data are generated from Whole Genome Sequencing (WGS) and Whole Exome Sequencing (WES) along with Aggregated Longitudinal Sequencing (ALseq) computational components. The transcriptomics data are generated from bulk RNA sequencing (bRNAseq) or single-cell RNA sequencing (scRNAseq). The proteomics data are generated from oligonucleotide-coupled or oligonucleotide-incorporated platforms (e.g. PEA-based proteomics via Olink, aptamer-based proteomics via SomaLogic, nELISA-based proteomics via Nomic Bio, or NULISA-based proteomics via Alamar Biosciences). The result of such analyses is integrated as a distinctive aspect of the LCM technique deployed.

The following sections shall now examine the aforementioned sub-components of the LCM analysis in details.

The above-mentioned Whole Genome Sequencing (WGS) and Whole Exome Sequencing (WES) techniques deployed for analyzing the genomic component of the multiomics process shall now be discussed in details.

Whole Genome Sequencing (WGS) provides a snapshot of the entire genome, including coding and non-coding regions, as well as structural variants leading to greater understanding of the genetic architecture and offering insights into genetic variations that may influence disease progression, treatment and therapeutic responses, whereby WGS is especially valuable in Longitudinal Capillary Multiomics (LCM), for the integration of diverse LCM data, providing a more complete view over time in observing occurrences of possible genetic alterations and aberrations due to disease progression, environmental factors, or therapeutic interventions. Moreover, Whole Exome Sequencing (WES) focuses exclusively on the protein-coding regions of the genome, which represent about 2% of the entire genome but harbor the majority of disease-associated mutations. This approach is highly effective in LCM application from a clinical perspective, to enable identification of specific genetic mutations linked to diseases, both in germline and somatic origins via WGS and WES, respectively.

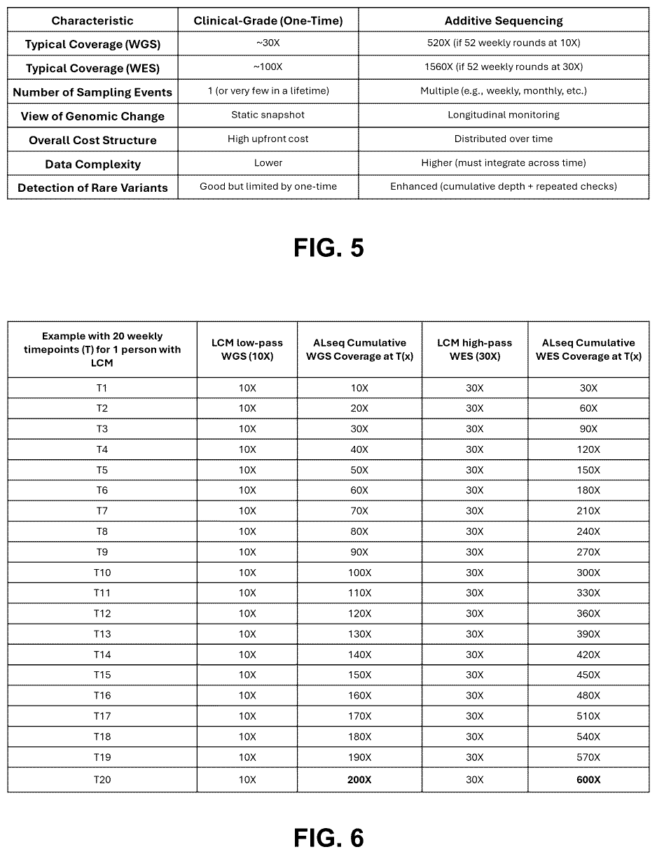

An example to illustrate the relevance of the WGS and WES shall help in elucidating the LCM genomic process further. Performing low-pass WGS at 10× coverage weekly in a year results in 52 time points and an aggregate coverage of 520×. This cumulative coverage provides significantly greater statistical power compared to a single high-coverage sequencing event. With reference to FIG. 5, by way of comparison and example, a one-time clinical-grade WGS might be 30×, while a one-time clinical-grade WES might be 100×. Even if a person were sequenced twice in their lifetime using the standard clinical approach, the cumulative coverage (60× for WGS or 200× for WES) would still be substantially lower than that offered by the proposed aggregated sequencing model of LCM. This massive difference in final coverage lays the groundwork for detecting low-frequency variants, structural rearrangements, and other genomic features that might be missed with conventional approaches.

Specifically, weekly low-pass whole-genome sequencing (WGS) at 10× coverage, repeated 52 times in a year, yields a cumulative 520× depth-far surpassing the one-time 30× often used in one-time clinical WGS. Likewise, weekly high-pass whole-exome sequencing (WES) at 30× coverage, conducted over 52 weeks, can cumulatively yield 1560×. Further, cumulative coverage enhances the ability to detect subtle but important genetic changes, which may otherwise be missed in a single sequencing event. Additionally, repeated sequencing allows tracking the emergence of mutations over time. For example, monitoring tumor mutational burden in real-time through LCM can provide valuable insights into how tumors evolve and respond to treatment. Similarly, in non-cancer diseases, longitudinal genomic data can reveal how mutations accumulate and potentially impact disease outcomes. Additionally, statistical reproducibility of WGS and WES is significantly enhanced as a consequence and feature of LCM, whereby repeated WGS and WES over time enhances reproducibility metrics.

With reference to FIG. 6, unprecedented detail about an individual's genetic landscape can be portrayed using the concept of aggregated longitudinal sequencing in genomics, wherein sequential genomic data is cumulatively combined to achieve exceptionally high coverage across multiple time points, longitudinal tracking of genomic variations, offering valuable insights into population health, biomarker discovery and disease evolution. The cumulative effects of ALseq are illustrated in FIG. 6.

Additive sequencing is anchored in the idea that repeated sequencing at moderate coverage can be aggregated to achieve ultra-deep coverage, thus increasing the sensitivity for variant detection. Mathematically, if Dᵢ represents the coverage depth at each time point i, then the cumulative coverage C(n) after n sequential time points is:

$$C ( n ) = {\sum }_{i = 1}^{n} Di$$

When each sequencing round is set to a fixed depth D, the total coverage after n rounds simplifies to:

$$C ( n ) = n \times D$$

In the illustrative scenario of weekly low-pass WGS at 10× for 52 weeks:

$${C}_{W G S} ( 52 ) = 52 \times 10 X = 520 X$$

Similarly, for weekly high-pass WES at 30×:

$${C}_{W E S} ( 52 ) = 52 \times 30 X = 1560 X$$

Regarding the aspect of analysis of genomic data over multiple time points, appropriate sequencing platforms provide an opportunity to track germline and somatic mutations in real-time. Moreover, the integration of low-pass whole genome sequencing (WGS) and high-pass whole exome sequencing (WES) within the Longitudinal Capillary Multiomics (LCM) framework offers a cost-effective strategy for frequent longitudinal analyses. The integration of WGS and WES in LCM is not only about tracking genetic changes but also of understanding how these changes can correlate with therapeutic states. As patients undergo treatment, pre- and post-therapeutic genomic changes can provide key insights into how therapy impacts the genome and vice versa. Understanding the successive mutational changes over long periods allows for better monitoring of treatment efficacy and safety monitoring.

The increased temporal resolution allows monitoring of subtle genetic changes, such as germline-to-somatic mutations, that may arise in response to disease progression, environmental exposure, or therapeutic interventions. More so, a cost-effective sequencing method enables combinatorial high-depth WES and low-depth WGS (or high-coverage WES and low-coverage WGS; or high-pass WES and low-pass WGS), providing a comprehensive genomic analysis with reduced expenses due to the fractional and aggregated longitudinal sequencing method of LCM. This approach aligns with the principles of LCM, offering a practical solution for longitudinal genetic studies.

The transcriptomics component of the LCM as stated above, involves gene expression that evolves throughout the course of a disease or therapeutic intervention, offering unprecedented comprehension into transcriptional profiles. An important characteristic of this process is the identification of different mRNA isoforms, resulting from alternative splicing events or other transcriptional modifications. These isoforms represent potential therapeutic targets where certain isoforms could be associated with disease progression or response to treatment, contributing to new possibilities for drug development. By way of example, a genome-wide analysis of aberrant RNA splicing in patients with acute myeloid leukemia (AML) can identify novel potential disease markers and therapeutic targets, which is considered a potent objective fulfilled. Also, alternative splicing is a common event in AML, involving many genes, with observance of different patterns of splicing in patients compared to normal patients, thereby highlighting the importance of alternative splicing in disease progression and its potential as a therapeutic target. LCM can be a powerful tool in understanding diseases in a similar manner.

Moreover, both bulk RNA-seq and single-cell RNA-seq have the capability to predict future clinical events based on mRNA signatures. For example, in clinical trial settings, LCM maps patient-specific gene expression patterns, identifying those who may respond poorly to certain treatments or are at risk of developing adverse effects. By monitoring mRNA changes in real-time, diagnostic experts can quickly identify which patients are benefiting from therapy and which may require adjustments to their treatment plans. This approach not only enhances treatment outcomes but also contributes to patient safety by detecting potential complications early on. In other examples, integrating single-cell and/or bulk RNA-seq identified prognostic signatures based on T-cell marker genes, which successfully predicted patient outcomes and therapeutic responses in lung squamous cell carcinoma.

In another aspect, biomarker discovery is a major strength of RNA-seq in the context of LCM as it enables the collection of genome-wide RNA data over time, facilitating the identification of novel transcripts that may serve as biomarkers for disease progression, treatment response, or disease susceptibility. This process is particularly important in clinical trials, where discovering new biomarkers can help identify patients who are most likely to benefit from specific therapies. Additionally, even in healthy populations, LCM can identify individuals at risk of developing certain diseases based on early changes in their mRNA profiles. The longitudinal nature of LCM ensures that these discoveries are made in a real-world context, providing more relevant, comprehensive and actionable data.

With regard to the proteomic analysis in the LCM process, it can be opined that Longitudinal Capillary Multiomics (LCM) integrates these measurements to offer a comprehensive view of disease progression, therapeutic responses, and underlying molecular mechanisms. Olink and SomaLogic are two such platforms that enable high-throughput proteomics with sensitivity and scalability, ideal for use in LCM.

In Olink's Proximity Extension Assay (PEA), employment of dual-recognition antibodies linked to DNA barcodes hybridize when bound to the same target protein, allowing for signal amplification and quantification via PCR and next-generation sequencing (NGS). This method enables the simultaneous measurement of up to 5,400 proteins per sample with exceptional specificity and sensitivity, making it ideal for longitudinal biomarker studies that require detecting subtle and dynamic changes in protein expression. Olink's PEA technology has been extensively applied in research on autoimmune diseases and cardiovascular biomarkers, offering valuable insights into inflammation and disease progression. For example, Olink's high-throughput proteomic platform could reveal plasma proteomic associations with genetic variants, emphasizing its role in identifying biomarkers for disease mechanisms and therapeutic response. Additionally, Olink's platform has uncovered associations between protein levels and cardiovascular diseases, thereby advancing the scope of understanding of complex diseases. This paragraph is referenced to highlight the use of oligonucleotide-coupled and oligonucleotide-incorporated labeling and detection chemistries for proteomics utility in LCM.

SomaLogic's Aptamer-Based Proteomics relies on synthetic single-stranded DNA aptamers, which bind target proteins with high affinity, allowing the quantification of up to 11,000+ proteins per sample. This extensive coverage enables a comprehensive profiling of disease-related pathways. It has been observed through various scientific studies that SomaLogic has contributed significantly to neurodegenerative disease research, identifying protein signatures associated with disease progression, which serve as potential biomarkers for early diagnosis and therapeutic intervention. In addition to Olink and SomaLogic, Nomic Bio and Alamar Biosciences proteomics platforms also include DNA-based integrations (i.e. oligonucleotides). This paragraph is referenced to highlight the use of oligonucleotide-coupled and oligonucleotide-incorporated labeling and detection chemistries for proteomics utility in LCM.

Further, the use of oligonucleotides-based chemistries in LCM is a distinct feature of LCM, whereby the genomics and transcriptomics components use oligonucleotides for probes and baits, and the proteomics component uses oligonucleotide integrations such as PEA-based chemistry, aptamer-based chemistry, nELISA-based chemistry, and NULISA-based chemistry. The oligonucleotide feature of the 3 omics within LCM enables the utility of combinatorial multiomics libraries (CML), whereby all of the sample preparatory workflows that lead to library generation that is characteristic of next-generation sequencing, can be combined on the same sequencing platform AND the same sequencing run AND the same sequencing flow cell. This enables further high-throughput efficiency for LCM due to the oligonucleotide component that is evident within LCM, whereby the concept of combinatorial multiomics libraries is realized.

Further, LCM also enables the use of repeated proteomic analyses over time, considered to be particularly important for monitoring dynamic changes in protein expression. Temporal proteomics allows detection of shifts in protein profiles that may not be apparent from a single sample. For example, longitudinal studies of cytokine profiles in diseases like rheumatoid arthritis have revealed key inflammatory pathways involved in disease progression, and these findings can help guide early intervention. Similarly, proteomic profiling during cancer therapy has provided insights into identifying biomarkers and signaling pathways after treatment.

The integration of LCM data is hereinafter discussed in these embodiments that deploys sophisticated computational tools (advanced statistical tools, e.g., R-Programming ecosysytem and the like) for analyzing the vast datasets generated by LCM, unifying complex data to reveal molecular connections that might be overlooked in single-omics analyses. Such integrative capability allows analysts to uncover connections between genetic differences, transcriptional modifications, and protein-level changes, providing deeper insights into abnormalities of the inner mechanisms of a human body and pathology.

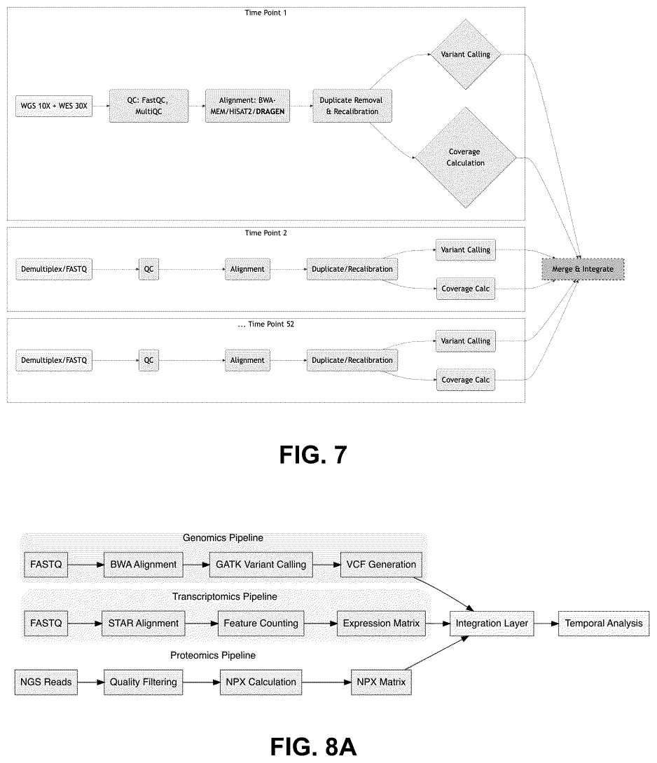

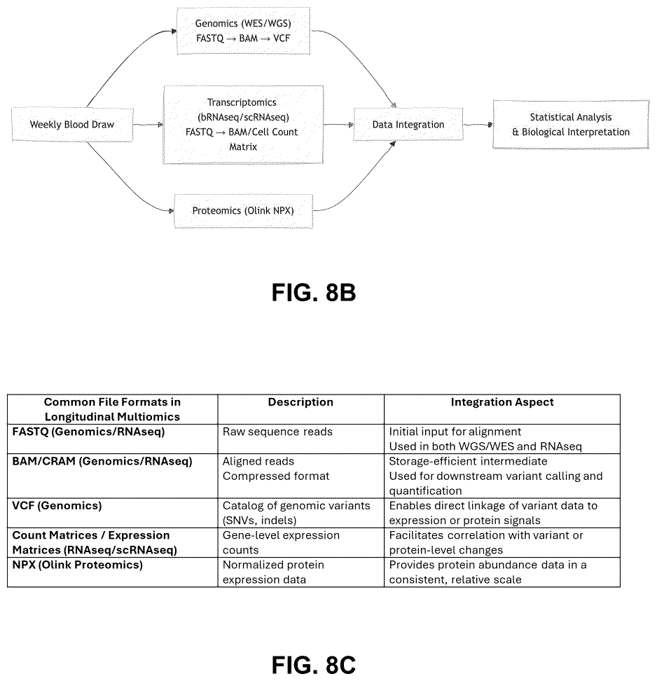

The practical computational analysis of the different multiomics data examined in LCM data analysis is now examined with respect to FIGS. 7, 8A, 8B, and 8C.

Genomics data generated from WES and WGS are typically stored in Variant Call Format (VCF) files. These files provide detailed information about identified genetic variants, including their genomic locations, reference alleles, and alternative alleles. VCF files consist of two main sections: a metadata header and a data section listing the specific variants.

With respect to transcriptomics analysis, data from bRNAseq typically comes in the form of count matrices, where rows represent genes and columns correspond to samples. These matrices serve as the foundation for downstream differential expression analysis. On the other hand, scRNAseq data is often stored in formats such as AnnData (in Python) or Seurat objects (in R). These formats not only include gene expression data but also metadata about cell types and experimental conditions, making them ideal for advanced analyses.

In R, proteomic data analysis typically starts with importing NPX files and applying normalization to correct for batch effects and technical variability. Tools like MSstats and OlinkAnalyze provide robust frameworks for analyzing proteomics data. MSstats offers a suite of statistical methods for differential expression analysis and visualization of protein data. Meanwhile, Olink Analyze is specifically designed to work with NPX data, streamlining tasks such as data import, normalization, and exploratory analysis.

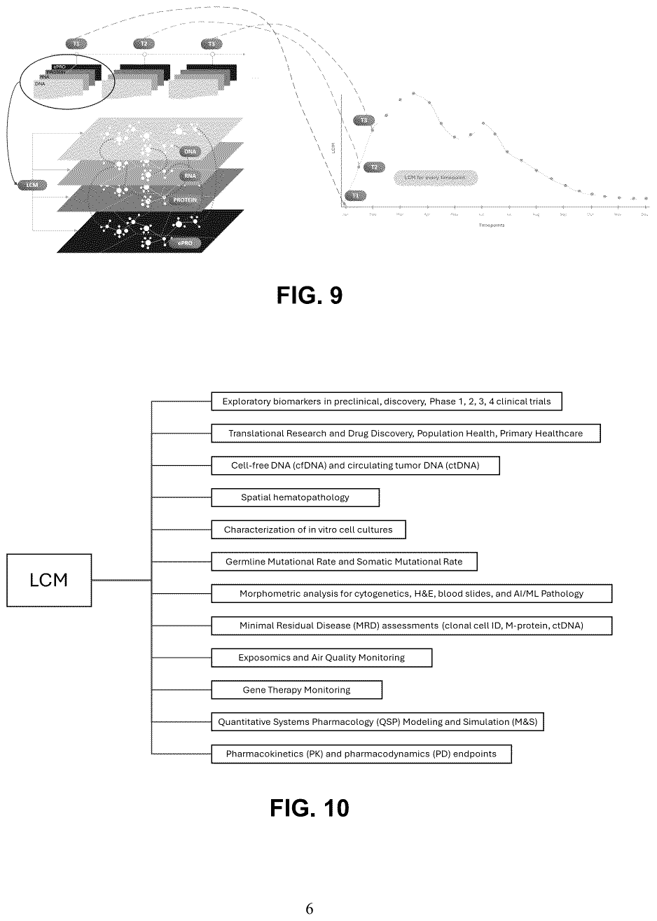

With reference to FIG. 9, the recording of the analyzed data in the LCM can be undertaken in a sequential and parallel format depending upon the requirement objectives, study design and available resources. While sequential processing examines each omics layer step-by-step, thereby maximizing the quality of individual datasets, it becomes especially useful when focusing on specific layers like proteomics or genomics, whereas parallel processing analyzes all omics layers simultaneously (i.e. genomics s+transcriptomics+proteomics), enabling synchronized and temporally aligned datasets ideal for time-series research. The process is standardized and automated using ML algorithms that converts and weaves raw data from LCM into insightful reports. Therefore, sequential and parallel processing of LCM data enables longitudinal, spatial, and temporal analysis.

Focusing on one layer at a time, whether genomics, transcriptomics, or proteomics, limits the scope of discovery since each omic layer works in different ways. For example, genomics can highlight mutations that influences protein-coding, but does not discretely identify proteins. On the other hand, transcriptomics displays active genes, but without proteomics, it is difficult to confirm whether these transcripts translate into active proteins. By merging these layers, Longitudinal Capillary Multiomics (LCM) not only provides a broader context but also enables a more precise molecular understanding of disease mechanisms and underlying biology.

Integrating transcriptomic and proteomic data has been instrumental in identifying post-transcriptional regulation mechanisms that influence tumor progression and metastasis. Alterations in regulatory sequences, RNA-binding proteins (RBPs), or upstream signaling pathways can affect mRNA stability and translation efficiency, thereby promoting tumorigenesis. For instance, RBPs play critical roles in the post-transcriptional regulation of gene expression, impacting processes such as pre-mRNA splicing, mRNA stability, polyadenylation, and translation. Dysregulation of these RBPs has been observed across various cancers, highlighting their potential as biomarkers and therapeutic targets. Meanwhile, the integration of genomics and proteomics in neurodegenerative diseases has facilitated the identification of somatic mutations that drive altered protein expression patterns, offering new targets for potential therapies. Also integrating genomic and proteomic data facilitates the identification of disease-specific genomic variants and their corresponding protein biomarkers. It emphasizes the role of proteogenomics in driving biomarker discovery and patient stratification.

Again with reference to FIG. 9, the LCM's ability to monitor multiple omic layers over time, at closer timepoints, provides a dynamic perspective of disease progression. For instance, repeated sampling at weekly time-points offers insights into short-term molecular fluctuations, while yearly sampling would miss these temporal nuances. This temporal resolution is critical, especially in rapidly progressing diseases or in response to treatment, as it enables researchers to observe real-time changes in gene expression, protein levels, and other molecular factors. This approach yields far more meaningful and actionable data compared to infrequent sampling, underscoring the importance of closely spaced time-points in LCM.

In another embodiment, this section examines the plurality of application areas where the above defined LCM process can be applied with reference to FIG. 10. These are being individually described along with their implication in detection and prediction of disease and utility:

Exploratory biomarkers in pre-clinical discovery, Phase 1, 2, 3, 4 clinical trials—Predicate observational studies led to accelerated R&D. For Phase 1, trials with LCM implementation can enable faster and more efficient biomarker data inclusive of genomics, transcriptomics and proteomics. Faster hypothesis generation can enable preclinical and discovery screening efficiency for putative therapeutic assets. Mechanism of action (MOA) studies can be accelerated via implementation of LCM, both in observational and open-label extension studies. For next-generation therapeutic asset planning, can be implemented directly into Phase 1 and 2 programs whereby open-label extension (OLE) studies introduce LCM as exploratory biomarker endpoints. Ongoing randomized clinical trials can include LCM as exploratory biomarkers to generate and facilitate translational biomarker discovery for potential asset development. LCM can also enable patient-specific targeted hypothesis generation for precision medicine efforts.

Translational Research and Drug Discovery, Population Health, Primary Healthcare—The pharmaceutical sector allows empowering translational biomarker teams with LCM to enhance primary and secondary endpoints with robust biomarker information via LCM as exploratory biomarker endpoints for validating exploratory assays in tandem with CDx and IVD development and discovering new pathways of disease and molecular signatures of response. Further, such pharmaceutical services enables therapeutic drug monitoring via harmonized proprietary LCM across all enrolled patients. The driving of personalized and precision diagnostics with proprietary LCM is enabled. The LCM technique also finds application by primary care providers. LCM addresses deficits in primary care for a defined population with a specific shared disease or medical condition, procedure, or care episode. Primary care providers/physicians (PCPs) gain more patient-specific disease intelligence from multiple longitudinal timepoints via LCM. Utilization of LCM empowers community health-workers with painless blood sample collection methods through innovative healthcare delivery models and addressing longstanding health and healthcare inequities in diagnostic access, whereby access to LCM is achieved in low-resource settings. LCM technique also finds application in clinical research and trial units for collaborative partnerships and research endeavors. LCM technique also finds application in various trials, e.g., in healthcare access for real-time multiomics in office by enrolling primary care practices and urgent care providers, exploratory multiomics pilots for longitudinal employee engagement and employee health in workforces, and advanced time-point honing for longitudinal exploratory translational enhancement for athletes.

Cell-free DNA (cfDNA) and Circulating Tumor (ctDNA) Analysis-LCM can demonstrate the value of disease screening and identification coupled to therapeutic drug monitoring of disease progression. Cell-free DNA (cfDNA), which includes circulating tumor DNA (ctDNA), can be viable for collection and stable recovery in upper arm capillary blood collection devices in less than 1000 μl of blood with high concentrations. Fragment sizing of cfDNA ranges from 50-300 bp, with 150 bp as average. Sizing of ctDNA included the range of cfDNA plus expanding up to 500-1000 bp or more in some examples. WES via LCM can be conducted on isolated cfDNA in both spiked and non-spiked samples, with applications related to non-invasive prenatal testing/screening (NIPT/NIPS) and oncology-related pathogenic variants (e.g. tumor mutational burden). Identification of aneuploidies, carrier screening, oncogenic variants, and tracking of specific variants can be confirmed in diseased and spiked samples. Changes in variants from longitudinal timepoints via LCM can be confirmed in cfDNA analysis, suggesting heterogeneity and successive clonal evolution of oncogenic drivers.

LCM MRD (capillary ctDNA)—MRD detects small fragments of circulating tumor DNA (ctDNA) within capillary blood samples for treatment efficacy, monitoring of disease progression, and post-therapeutic long-term monitoring of recurrence. Proprietary longitudinal timepoints via LCM enables robust monitoring by using ctDNA as the specific biomarker of interest for early detection and active surveillance strategies. Further, LCM MRD enables matching of patients to effective targeted treatments and personalized medicine strategies at earlier interventional stages due to the ease of collection and ctDNA analysis via the discussed LCM process. By focusing exclusively on ctDNA, enhanced coverage and reads for capillary ctDNA is achieved by novel isolation of ctDNA within capillary samples followed by WGS, WES, or WTS, with preferential coverage and read allocation for known oncogenes of interest (e.g. TSO500 panel). LCM MRD also enables tumor microenvironment molecular characterization by using capillary samples as a proxy over multiple timepoints. LCM MRD can also measure neoadjuvant therapy response, overall MRD and burden, monitor adjuvant therapy response over time, and monitor ctDNA levels for recurrence. Furthermore, LCM MRD provides quantitative capillary ctDNA parts per million (PPM) scores in addition to adjacent transcriptomics and proteomics data as ad-hoc supplementary data. The capillary ctDNA is devoid of genomic DNA, whereby the ctDNA is further sequenced (WGS and/or WES). LCM MRD can achieve ctDNA early detection (e.g. early ~130-180 days) in advance of radiographic data.

Spatial Hematopathology Transcriptomics—LCM can be enhanced with single-cell molecular characterization with longitudinal morphological retention of “dropped” blood miscroscope slides over time-series for mRNA ISH transcripts that retain morphology for the end-user. Highly multiplexed panels for immune-checkpoints and pan-cancer genes, all for mRNA transcripts, can achieve distinct morphological resolution via automated digital pathology single-cell molecular characterization of PBMCs over time to enable longitudinal profiling (e.g. track clonal heterogeneity, heterogenous PBMC populations, and clonality over time), which is amenable for pre- and post-therapeutic monitoring of blood disorders. Therefore, LCM coupled to morphological assessments (e.g. mRNA ISH and MERFISH) of blood is an example application.

In-vitro cell culture analysis of blood cells, stem cells, and reprogrammed cells—The optimized protocols shall facilitate physiological and cellular characterization of these processes, which may be used for fast screening of specific therapeutic cancer drugs for migratory function, novel strategies in cancer diagnosis, and for assaying new molecules involved in adhesion and invasion of metastatic properties of cancer cells. LCM can be utilized for such in vitro cell cultures.

Germline Mutational Rate (GMR) and Somatic Mutational Rate (SMR)—The LCM process can enhance traditional germline mutational analysis to predict the future course and likelihood that an individual will develop disease. Somatic Mutation Rate (SMR) identifies the rate of change over a lifelong longitudinal assessment as compared to germline (constitutional baseline) and all subsequent somatic timepoints. SMR shows a significantly higher rate of change as compared to the germline mutational rate (GMR). Successive timepoints confirm progressive rates over time. Whole genome sequencing (WGS) and whole exome sequencing (WES) via LCM can be deployed across various coverages (e.g. at 5, 10, 20, 30, 50 or 100×; anywhere between 1-100×) from upper arm capillary whole blood collections per LCM. LCM enables comparisons of both standard and rare germline variation within the entire human genome set of diverse genes that commonly impacts mutational processes in somatic cells. Given that somatic mutations are components of the biological and cellular ageing process, tracking of the changes in somatic mutations (i.e. SMR) can demonstrate the rates and types of mutations across individuals and populations as a feature of LCM. This can be used to determine disease progression. Associations between deleterious variants and somatic mutations within longitudinal timepoints can provide evidence of gene interactions that drive mutagenesis. The LCM SMR application is used to identify the variant rate of change and longitudinal disease signatures within individuals by implementing standardized PICARD analysis across every longitudinal timepoint. Therefore, LCM for germline mutational rate and somatic mutation rates is one such application.

Morphometric analysis of cytogenetics, H&E, blood slides and AI/ML Digital Pathology—LCM can enhance morphometric analysis by augmenting non-morphometric data (i.e. LCM data) with morphometric data (i.e. cytogenetics FISH, H&E stains, digital pathology). In this example, LCM incorporates longitudinal morphometric data from the same capillary whole blood collections that are achieved within the LCM process. Morphometric data from the same LCM timepoints is achieved via preparation of the same capillary blood into prepared microscope slides for blood analysis. Pairing LCM with morphometric (i.e. visual) biological analysis is one such application.

Minimal Residual Disease (MRD) assessments (Clonal cell ID, M-protein, ctDNA)—LCM MRD can be achieved as a result of longitudinal collections. In this case, such longitudinal collections entail capillary blood collections, as described in the LCM process, whereas traditional MRD assessments incorporate venipuncture whole blood collections from venous blood draws. Therefore, LCM enables MRD via clonal cell ID tracking (i.e. the tracking of VDJ immunophenotypes within the capillary blood collection), M-protein tracking (i.e. the quantitative assessment of M-protein that is malignantly produced in some cancers which can be detected in blood serum and blood plasma), and ctDNA (as described above in prior paragraphs).

Exposomics and Air Quality Monitoring-Exposomics is enabled via, whereby one example application is the utility of LCM (molecular data) coupled to radon counts (environmental data), enabling robust exposomics monitoring in a longitudinal manner. Radon-induced lung cancer is the second leading cause of lung cancer in non-smokers, with additional significant HR/OR/RR that includes childhood leukemias. Environmental monitoring of radon across US homes is limited. LCM can enable real-time monitoring of radon-exposure tethered to robust multiomics biomarker assessments via the LCM process for inhabitants (families) of single-family homes. Proprietary time-points based LCM process for in-home radon detection offers real-time, high-sensitivity molecular characterization of environmental exposure (radon) via longitudinal capillary multiomics over multiple timepoints for families living in radon-afflicted homes.

Gene Therapy Monitoring—LCM can be implemented as a new methodology to assess the potential pathogenicity of replication competent retrovirus (RCR) and replication competent lentivirus (RCL). For retroviral vector-based gene therapy patients, LCM can be implemented for presence/absence studies to detect the presence in RCR, whereby molecular characterization profiles via LCM can be developed for both binary states. Additional correlation assessments between LCM and insertion-site analysis (ISA) can be conducted for orthogonal confirmation, whereby LCM can determine the exact location and quantity of positively-identified vector insertion sites within PBMCs directly from capillary blood samples. LCM can serve as a RCR detection and quantitation assay for lifelong monitoring of cell and gene therapy patients as an exploratory endpoint and safety and monitoring endpoints. LCM can also be conducted on the supernatants of cell therapy manufacturing products in addition to capillary blood samples post-therapy of manufactured products. cPBMCs can be isolated via proprietary LCM processes followed by the LCM process for RCR-specific signatures. Confirmation can be obtained via genomics as part of the proprietary LCM process, with additional biological insights of pathways as identified by the remainder omics of the LCM workflow. Therefore, LCM can be an efficient method for exploratory and supplemental monitoring of gene therapies for lifelong time-series and adverse-event monitoring needs.

Quantitative Systems Pharmacology (QSP) Modeling and Simulation (M&S) and Pharmacokinetics (PK) and Pharmacodynamics (PD)—QSP can be achieved via LCM by integrating computational modelling with experimental data to elucidate complex biological and pharmacological systems. By simulating drug behavior and interactions within biological networks, QSP enables researchers to predict therapeutic efficacy, optimize dosing regimens, and minimize adverse effects. The growing complexity of drug development and the increasing demand for precision medicine have amplified the need for robust data inputs to power QSP models effectively, whereby LCM can be implemented to fill the data inputs via robust multiomics gained from longitudinal timepoints. Therefore, LCM QSP can facilitate the understanding of mechanisms of action for both new and existing drugs, maximizes therapeutic benefits, reduces toxicity, and establishes strategies to improve individual patient outcomes. By leveraging mechanistic mathematical models, LCM QSP can characterize the dynamic interactions between drugs and physiological processes, offering insights into biological systems at multiple levels of organization from molecular and cellular to organ-level networks. This multi-scale approach empowers researchers to analyze and predict the complexities of drug interactions within the human body, solidifying LCM QSP as a cornerstone of precision pharmacology. Central to the success of LCM QSP is the ability to collect high-quality, longitudinal biomarker data that captures dynamic physiological changes over time. Traditional methods of blood sample collection, which often rely on venipuncture, present challenges such as patient discomfort, logistical hurdles, and limited feasibility for frequent sampling. These limitations hinder the acquisition of dense time-series datasets, a critical requirement for accurate pharmacokinetics (PK) and pharmacodynamics (PD) studies. Therefore, LCM QSP is one such application of LCM.

What is claimed is:

1. A longitudinal capillary multiomics (LCM) method and system for analyzing biological samples, the method comprising:
Collecting capillary blood micro-samples from the upper arm of subject in a minimally invasive manner over space and time;
Subjecting the blood samples to Longitudinal Capillary Multiomics (LCM) analysis comprising the sub-processes of genomics, transcriptomics and proteomics; and
Integrating and analyzing the LCM data of genomics, transcriptomics and proteomics for multiomics assessments and exploratory biomarker analysis;
Wherein the said analysis of LCM towards creating a longitudinal biomarker profile of a subject involves Whole Genome Sequencing (WGS), Whole Exome Sequencing (WES), Aggregated Longitudinal Sequencing (ALseq), bulk RNA sequencing (bRNAseq), and oligonucleotide-incorporated or oligonucleotide-coupled labeling and detection platforms for high-throughput proteomics.

2. The method of claim 1, wherein the said capillary blood samples of subjects is collected remotely, over space and time, from upper-arm of subjects through blood collection devices for conducting a temporal LCM analysis.

3. The method of claim 1, wherein the said whole genome sequencing (WGS), whole exome sequencing (WES) and aggregated longitudinal sequencing (ALseq) within the genomics component of the Longitudinal Capillary Multiomics (LCM) framework generates longitudinal data across multiple time points, facilitating the tracking of genetic changes over time.

4. The method of claim 3, wherein the said Whole Genome Sequencing (WGS) provides a snapshot of the entire genome, leading to greater understanding of the genetic architecture and offering insights into genetic variations that may influence disease progression, treatment resistance and therapeutic responses.

5. The method of claim 3, wherein whole exome sequencing (WES) provides a more complete view of the occurrence of possible genetic alterations due to disease progression, environmental factors, or therapeutic interventions over time.

6. The method of claim 3, wherein the integration of low-pass whole genome sequencing (WGS) and high-pass whole exome sequencing (WES) within the Longitudinal Capillary Multiomics (LCM) framework offers a cost-effective strategy for frequent longitudinal analyses.

7. The method of claim 1, wherein the aggregated longitudinal sequencing method in the genomic analysis entails low-pass whole-genome sequencing that is aggregated over time to create an additive and cumulative effect of the sequencing coverage.

8. The method of claim 1, wherein the aggregated longitudinal sequencing method in the genomic analysis entails high-pass whole exome sequencing that is aggregated over time to create an additive and cumulative effect of the sequencing coverage.

9. The method of claim 1, wherein the concept of additive and aggregated sequencing in the genomics component of LCM is achieved by longitudinal repeated sequencing that is aggregated over time to achieve ultra-deep coverage, thereby increasing the sensitivity for genetic tracking of variants and genomic aberrations.

10. The method of claim 1, wherein the data from the transcriptomics component of LCM involves bulk RNA sequencing (bRNAseq) for homogenous gene expression RNA profiling.

11. The method of claim 1, wherein the data from the transcriptomics component of LCM can additionally include single-cell RNA sequencing (scRNAseq) for heterogenous RNA profiling.

12. The method of claim 1, wherein the said RNA-seq in the context of the transcriptomics component of LCM enables collection of genome-wide RNA data over time, facilitating the identification of novel transcripts serving as biomarkers for disease progression, treatment response, disease susceptibility, or biomarker health status.

13. The method of claim 1, wherein the proteomics component of the LCM utilizes platforms like Olink, SomaLogic, Nomic Bio or Alamar Biosciences for enabling high-throughput proteomics of a subject with sensitivity and scalability along a temporal domain.

14. The method of claim 1, wherein the proteomics platform involves oligonucleotide-incorporated or oligonucleotide-coupled labeling and detection chemistries, such as proximity extension assay (PEA)-based technology, aptamer based technology, nELISA-based technology, and NULISA-based technology, such that the oligonucleotide feature enables consolidated integration into high-throughput sequencing platforms and flow cells.

15. The method of claim 1, wherein repeated proteomic analyses over time, for monitoring dynamic changes in protein expression allows detection of shifts in protein profiles that may not be apparent from a single sample.

16. The method of claim 1, wherein the integration of LCM data using statistical tools allows room for uncovering connections between genetic differences, transcriptional modifications, and protein-level changes, providing deeper insights into abnormalities of the inner mechanisms of a human body and cellular processes.

17. The method of claim 1, wherein the recording of the analyzed data in the LCM can be undertaken in a sequential or parallel format depending upon the requirement objectives, study design and available resources.

18. The method of claim 17, wherein sequential processing examines each omics layer step-by-step and over longitudinal timepoints, thereby maximizing the quality of individual datasets, whereas parallel processing analyzes all omics layers simultaneously, enabling synchronized and temporally aligned datasets ideal for time-series research and longitudinal assessments.

19. The method of claim 1, wherein the LCM process is standardized and automated using artificial intelligence (AI) and machine learning (ML) algorithms for converting, integrating, and weaving raw data from LCM analysis into insightful and automated reports.

20. The method of claim 1, whereby the plurality of application of LCM includes, but not limited to, services in the pharmaceutical sector and clinical research organizations, exploratory biomarker endpoints in pre-clinical discovery, Phase 1, 2, 3, 4 clinical trials, translational research and drug discovery, population health, primary healthcare, cell-free DNA (cfDNA) and circulating tumor (ctDNA) analysis, molecular and minimal residual disease (MRD) assessments by clonal cell ID, M-protein, and ctDNA, spatial hematopathology, in vitro cell culture analysis of blood cells, stem cells, and reprogrammed cells, germline mutational rate (GMR) and somatic mutational rate (SMR), morphometric analysis of cytogenetics, H&E, blood slides and AI/ML digital pathology, exposomics and air quality monitoring, gene therapy monitoring, quantitative systems pharmacology (QSP) modeling and simulation (M&S) and pharmacokinetics (PK) and pharmacodynamics (PD) assessments, and primary healthcare access for real-time longitudinal multiomics.
